(19) United States
(12) Patent Application Publication
(10) Pub. No.: US 2020/0126659 A1
(43) Pub. Date: Apr. 23, 2020

(54) METHOD AND SYSTEM FOR QUALITY CONTROL SYSTEM AND FRAMEWORK FOR AN EVIDENCE-BASED ORGANIZATION

(71) Applicants: Mary K. O'Fallon, Denver, CO (US); Kammie Monarch, Oklahoma City, OK (US); Mary Beth Wenger, Oklahoma City, OK (US)

(72) Inventors: Mary K. O'Fallon, Denver, CO (US); Kammie Monarch, Oklahoma City, OK (US); Mary Beth Wenger, Oklahoma City, OK (US)

(21) Appl. No.: 16/694,480

(22) Filed: Nov. 25, 2019

Related U.S. Application Data
(63) Continuation of application No. 14/431,195, filed on Mar. 25, 2015, now abandoned.
(60) Provisional application No. 61/705,479, filed on Sep. 25, 2012.

(51) Int. Cl. G16H 40/20; G06Q 10/06; G06Q 50/22

(52) U.S. Cl. CPC G16H 40/20; G06Q 50/22; G06Q 10/0639

(57) ABSTRACT

A quality control system and framework for an evidence-based organization are disclosed herein. The present disclosure relates to methods and systems for training, implementing and measuring outcomes, as well as techniques for improving outcomes of home visitation and consultation services provided by medical practitioners to, for example, first time parents and their families.

### CROSS REFERENCE TO RELATED APPLICATIONS

The present application is a continuation application of U.S. Non-Provisional application Ser. No. 14/431,195, filed Mar. 25, 2015, which is a national stage application under 35 U.S.C. § 371 of International Application No. PCT/US2013/061772, filed Sep. 25, 2013, and claims priority to U.S. Provisional Application No. 61/705,479, filed Sep. 25, 2012, and entitled “Method and System for Quality Control System and Framework for an Evidence-Based Organization,” all of which are hereby incorporated by reference herein in their entireties.

### TECHNICAL FIELD

The present disclosure relates to systems and methods for quality control and improvement. More specifically, the present disclosure relates to methods and systems for training, implementing and measuring outcomes of home visitation and consultation services provided by medical practitioners to, for example, first time parents and their families.

### BACKGROUND

In order to receive and sustain both government and private funding, evidence-based organizations, which includes both for profit and not-for-profit entities, are often required to show that their initiatives for which they received funding are successful and achieve the stated goals or outcomes. However, metrics by which an evidence-based organization is deemed successful are often defined by each individual organization and may lack an objective standard. Accordingly, a true assessment of whether an organization is succeeding in helping its target population is difficult to ascertain. Further, an organization may struggle to establish its own independent, verifiable criteria to measure success and outcomes and to define specific areas in need of improvement.

The information included in this Background section of the specification, including any references cited herein and any description or discussion thereof, is included for technical reference purposes only and is not to be regarded subject matter by which the scope of the invention is to be bound.

### SUMMARY

One aspect of the subject matter of the present disclosure addresses the aforementioned needs by providing a method and system for an organization, such as an evidence-based organization, to develop and implement a quality framework plan and quality control program.

In one aspect, a method for implementing a quality control and improvement program in an evidence-based organization is disclosed. The evidence-based organization may be an organization established to help low-income, first time mothers, pregnant mothers, infants and young children. In one aspect, the method includes evaluating an implementation plan and feasibility assessment and incorporating those findings into an annual plan for the organization. The method may further include applying or implementing the annual plan within the evidence-based organization. The evidence-based organization may also provide educational training during or after the initial evaluation step. In addition, the method may include providing assessment surveys to stakeholders of the evidence-based organization. The stakeholders may include a first-time mother, a pregnant mother, infants, young children, and home visitors, such as a nurse or other health professional or an employee of the evidence-based organization. Raw data is collected from the assessment surveys and analyzed to identify action items for improvement by the stakeholders and by the evidence-based organization. The evidence-based organization may implement the action items to improve quality for the current stakeholders. The evidence-based organization may implement the action items to develop and improve a quality control program for the benefit of future stakeholders.

In some aspects, the assessment surveys may include at least one of client interaction surveys, program implementation surveys and outcome achievement surveys. The implementation plan may include data related to at least one of: (i) characteristics of a community to be served by the evidence-based organization; (ii) budget and funding mechanisms; (iii) a timeline for implementation; and (iv) identification of challenges expected by the evidence-based organization in its implementation. The feasibility assessment may include data related to at least one of: (i) identification of one or more needs of a community to be served by the evidence-based organization; (ii) evidence of a viable client referral network; and (iii) presence of community resources to support referrals made by the evidence-based organization. In one aspect, the analyzing operation further includes comparing the response score or the overall score to a benchmark or a target score. In one aspect, the method further includes evaluating the overall score by determining where the score falls in a range of scores, wherein the range provides an indication of a performance level of the evidence-based organization or the stakeholders. The performance level includes high performing, performing, low performing or critically low performing. In some aspects, the method further includes developing and implementing an improvement plan for an evidence-based organization or stakeholders having a performance level of low performing or critically low performing.

In one aspect, a system for implementing a quality control and improvement program in an evidence-based organization is disclosed. The system includes a computer having a memory for storing computer readable code and a processor operatively coupled to the memory. The processor is configured to receive raw data from assessment surveys given to stakeholders of the evidence-based organization, wherein the stakeholders comprise at least one of a first-time mother and a nurse; analyzing the raw data from the assessment surveys to determine a score for each response in the survey; and sorting and weighting each response score to determine an overall score. The overall score is utilized to identify action items for improvement for the stakeholders and further to identify action items for improvement for the evidence-based organization as a quality control program. In one aspect, the computer is a special purpose computer. In one aspect, the evidence-based organization is a home visitation organization established to help low-income, first time mothers, pregnant mothers, infants, young children and home visitors. In one aspect, the assessment surveys include at least one of client interaction surveys, program implementation surveys and outcome achievement surveys. In one aspect, the analyzing operation further comprises comparing the response score or the overall score to a benchmark or a target score. In one aspect, the system further includes evaluating the overall score by determining where the score falls in a predetermined range, wherein the predetermined range provides an indication of a performance level of the evidence-based organization.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Other features, details, utilities, and advantages of the present invention will be apparent from the following more particular written description of various embodiments of the invention as further illustrated in the accompanying drawings and defined in the appended claims.

### BRIEF DESCRIPTION OF THE DRAWINGS

The present disclosure, both as to its organization and manner of operation, may be understood by reference to the following description, taken in connection with the accompanying drawings, in which:

FIG. 1 is a flow chart illustrating a method for developing and implementing a quality control framework according to aspects of the present disclosure;

FIG. 2 is a flow chart illustrating aspects of the pre-implementation module of the method of FIG. 1;

FIG. 3 is a flow chart illustrating aspects of the training module of the method of FIG. 1;

FIG. 4 is a flow chart illustrating aspects of the “launch to first year” module of the method of FIG. 1;

FIG. 5 is a flow chart illustrating aspects of the “year 2” module of the method of FIG. 1;

FIG. 6 is a flow chart illustrating aspects of the “year 3 and beyond” module of the method of FIG. 1;

FIG. 7 is a flow chart illustrating aspects of the data analysis utilized by the method of FIG. 1 and which may be performed by a computer system as disclosed herein;

FIG. 8 is a block diagram of a computer system that may be used for implementing aspects of the method of FIG. 1, including aspects of FIG. 7; and

FIGS. 9A-9E depict one embodiment of a user interface of the system of FIG. 8.

### DETAILED DESCRIPTION

Formalized quality improvement programs are new to the home visitation industry. Home Visitation for pregnant women, infants and young children is designed to support healthy birth outcomes, infant and child health and development and positive maternal life course outcomes. Data analysis and quality improvement are each aspects of the model and the formalized process (model) described herein describes criteria to measure, analyze, and use data to improve the home visitation program at the individual agencies and for the home visitation program (model) overall.

Implementing a quality control program and/or a quality framework is one method of assessing both the performance of an organization and the outcomes of the intervention. A quality framework may also include methods to improve performance. Under one definition, quality may be defined as the degree to which program implementation occurs as designed, interventions meet model fidelity and outcomes are achieved.

Disclosed herein is a method and system for developing and implementing a quality framework for an organization, such as an evidence-based organization. In one embodiment, the method may include a formal approach to assess performance and the use of systematic methods to improve processes and outcomes. Advantages and characteristics of the disclosed method include: (1) the process is based on data; (2) the process is participatory; (3) the process uses both prospective and retrospective review; (4) the process is transparent; (5) the process is aimed at improvement; (6) the process uses strategies based on data analysis and testing; and (7) the process draws from a convenience sample and review; it does not have to be randomized or controlled.

For the convenience of the reader, the remainder of this disclosure is made in terms of an evidence-based organization that supports home visitation of first-time mothers and their children. It can be appreciated that other evidence-based organizations may also use the methods and systems disclosed herein. The organization may also help other organizations develop and implement quality control and quality framework methods as disclosed herein. Such organizations may receive funding from federal, state and local governments as well as from independent funders. The organization may have various stakeholders, such as clients and the medical professionals that serve their needs. Its clients may be interchangeably referred to as a “client” or as a “first time mother”. The medical professionals may include nurses, who may be referred to as nurse home visitors, nurse supervisors and nurse consultants. In an embodiment where a first organization is overseeing the development and implementation of a quality framework for a second organization, the nurse consultant is an employee of the first organization and the nurse supervisor and nurse home visitors are employees of the second organization. Quality assessments occur at the client, nurse and agency (second organization) levels. The nurse consultant (first organization) collaborates with the agency administrator (second organization), nurse supervisors and nurse home visitors at the site and state levels to review data and plan and implement quality improvement strategies in an on-going manner.

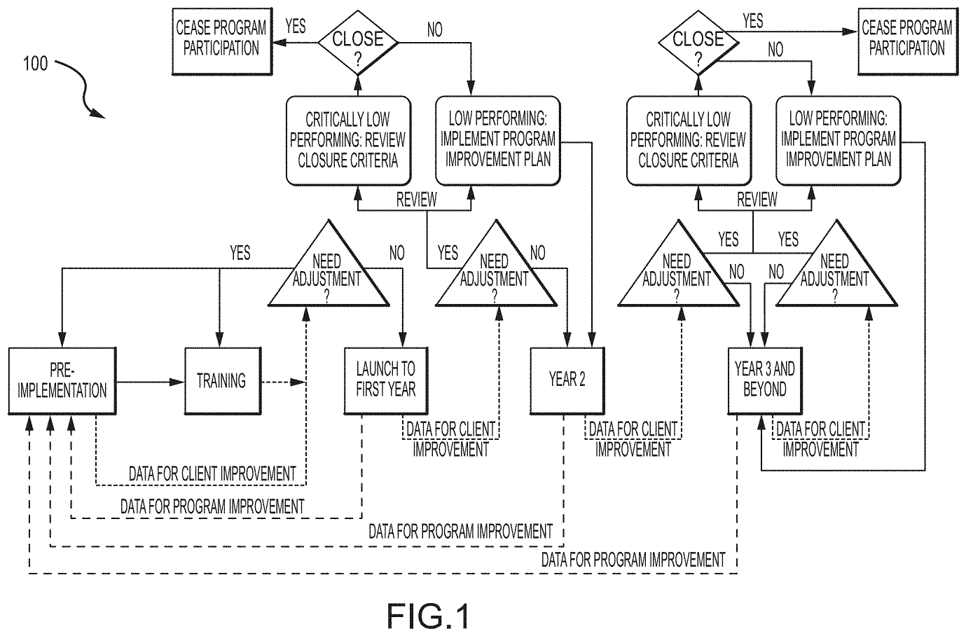

In one aspect of the method, and with reference to FIG. 1 which is a flow chart illustrating one embodiment of a method 100 for developing and implementing a quality control program and framework, participation in the quality framework begins when an agency (second organization) submits its implementation plan to the first organization for review. Measurement and quality improvement occur in implementation phases, operations or modules: pre-implementation; launch-year one; years two to three; and years three and beyond. As can be understood from FIG. 1, data is collected during each phase and is used going forward to improve services for the particular client and is used retrospectively to improve quality of (services offered by) the agency. The agency may evaluate and analyze data and reports generated from the various tools and assessments and the first organization may use this information to improve processes at the first organization, such as education and consultation. The data may be analyzed as disclosed elsewhere herein and may be used to determine whether an agency is performing at a high standard or whether improvements are needed or required. If no adjustments are needed, the agency may proceed to the next year. If adjustments are needed, a program improvement plan may be implemented to help the agency perform better in the following year. If an agency is critically low performing, the first organization may review the closure criteria and determine whether the agency should cease operations or implement a program improvement plan to help performance for the next year. While the disclosed embodiments are described in specific terms, other embodiments encompassing principles of the invention are also possible. Further, operations may be set forth in a particular order. The order, however, is but one example of the way that operations may be provided. Operations may be rearranged, modified, or eliminated in any particular implementation while still conforming to aspects of the invention. Accordingly, the operations of the method 100 may be performed in the order illustrated, in another suitable order and/or one or more operations may be performed simultaneously. Moreover, in some embodiments, the method 100 may include more or fewer operations than those illustrated.

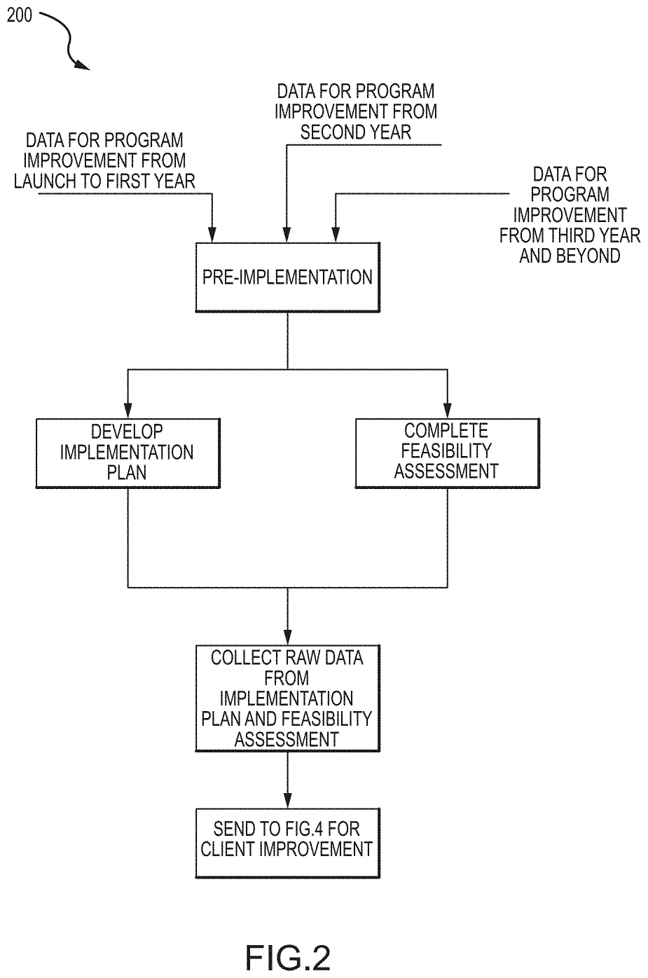

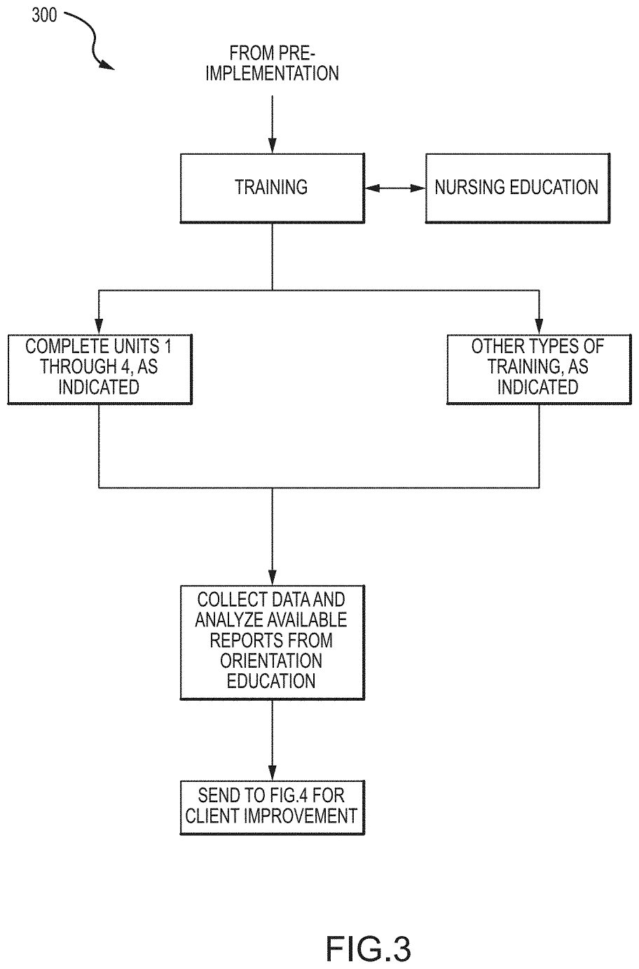

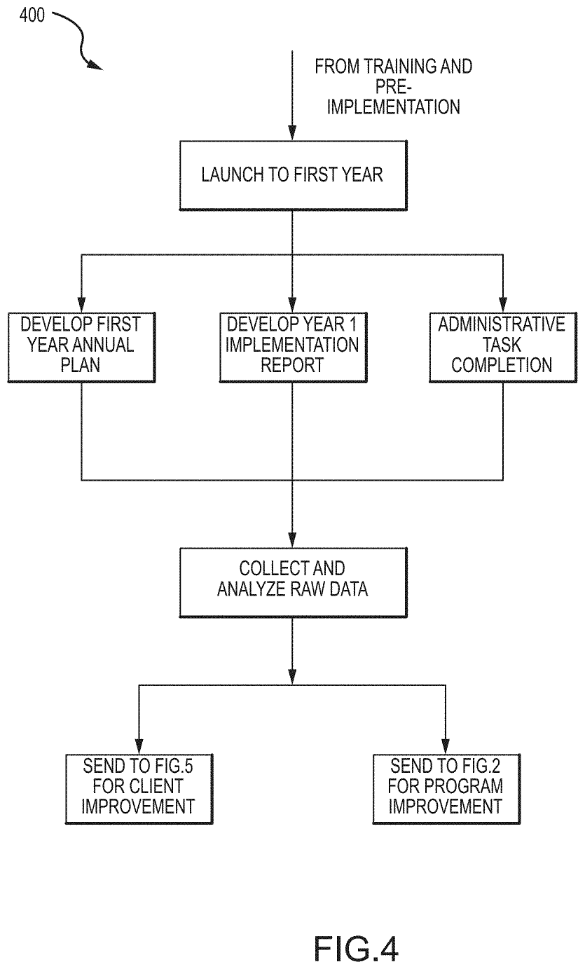

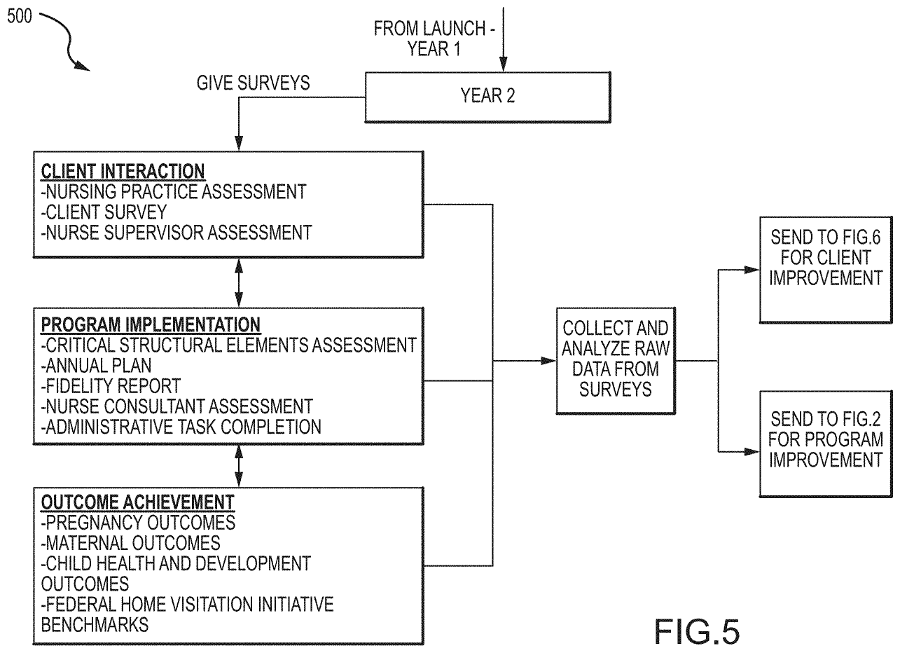

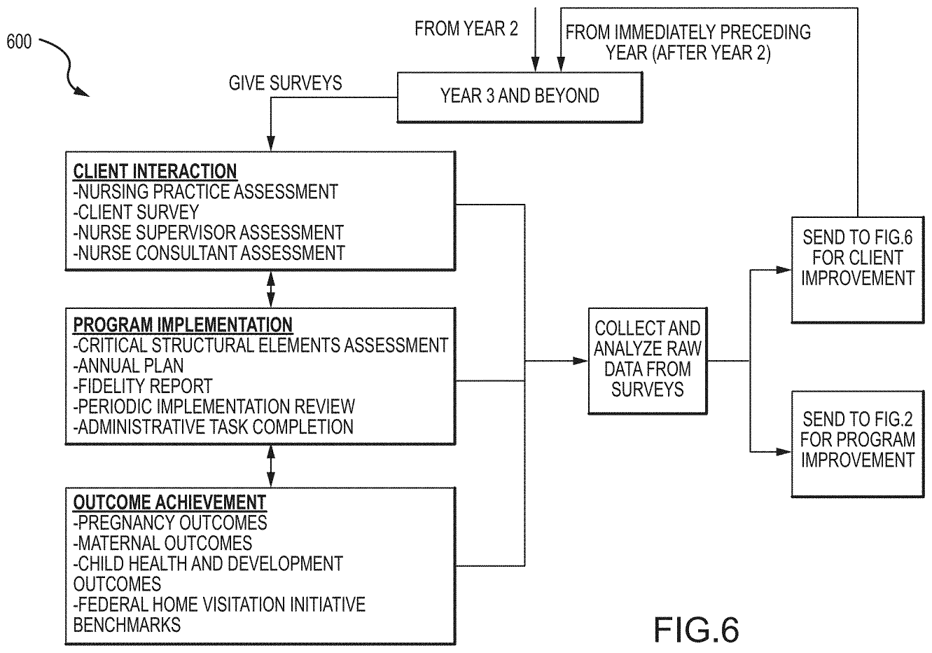

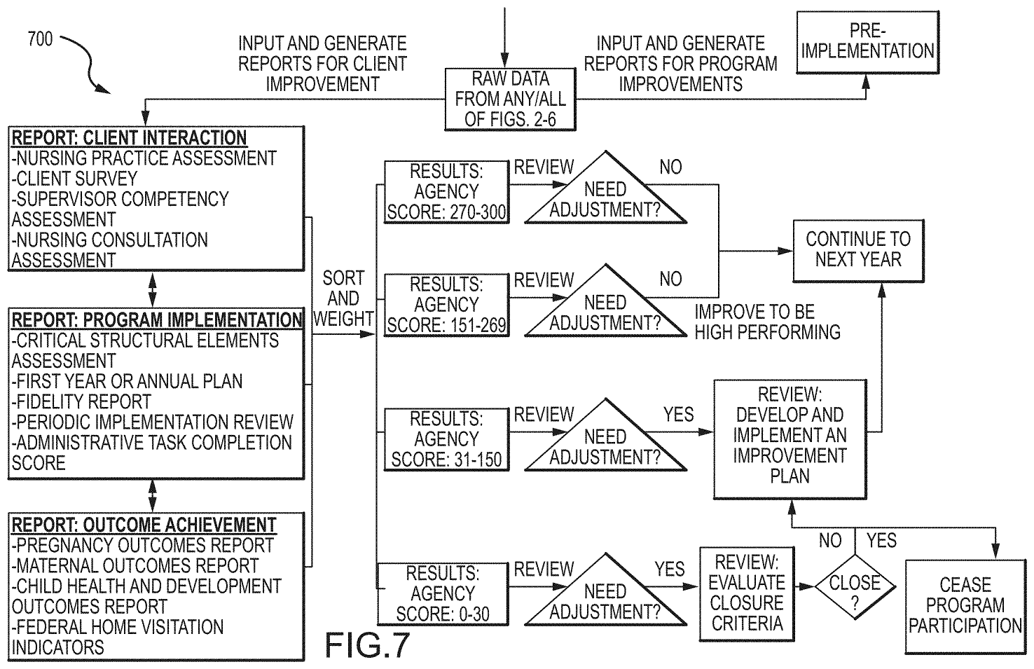

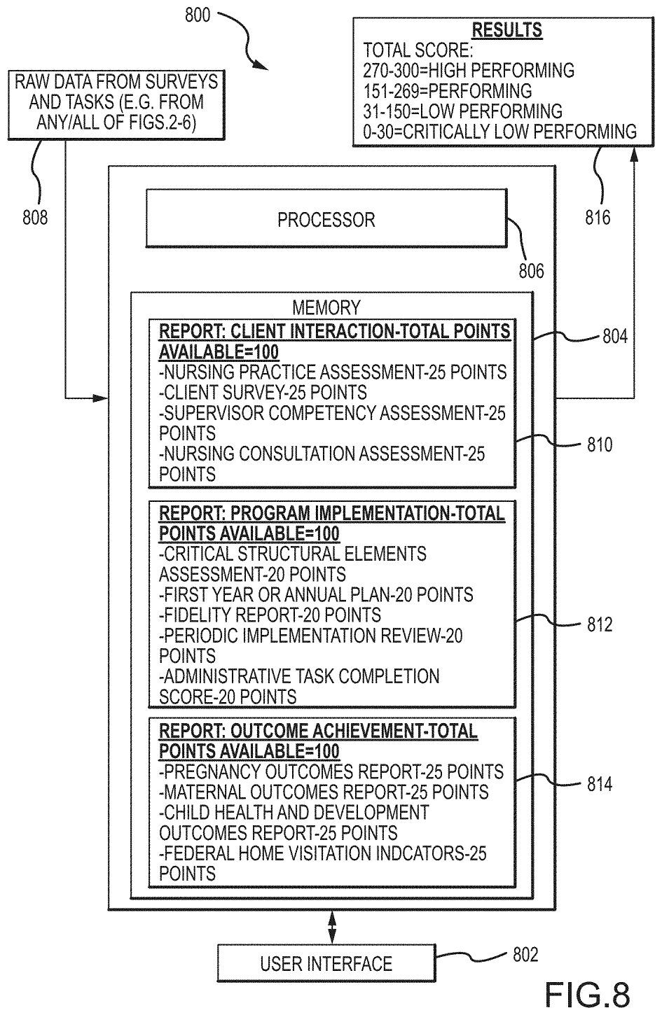

Aspects of the pre-implementation module 200 are illustrated in the flow chart of FIG. 2. As shown in FIG. 2, the pre-implementation module 200 includes review and development of an agency's implementation plan and completion of a feasibility assessment. Data is collected and analyzed and utilized to improve quality of the client program in the launch to first year module. Data for Program Improvement from each of the “Launch to First Year” module, “Year Two” module, and “Year Three and Beyond” modules is collected, analyzed and utilized to improve quality of the agency's program implementation and/or the first organization's program quality framework. In one embodiment, the Implementation Plan is a detailed template into which a program developer, such as a program developer employed by the first organization, and a prospective implementing agency representative may enter the following information, including, but not limited to: the community population characteristics; the agency's nurse recruitment and hiring practices; evidence of sufficient political support and advocacy influence to sustain the economics of the program; budget and funding mechanisms; a timeline for implementation and successful caseload ramp-up and an outline of relevant challenges. The Feasibility Assessment is completed at the same time as the Implementation Plan. The Feasibility Assessment is a template completed by the prospective implementing agency and the first agency's program developer and provides an environmental scan including demonstrated community need for the intervention, evidence of a viable client referral network and presence of community resources that will support home visitation referral needs such as mental health agencies.

Aspects of the training module 300 are illustrated in the flow chart of FIG. 3. As shown in FIG. 3, the training module includes completion of at least one of units one through four of the orientation education, as may be indicated, completion of nursing education if needed and any other types of training that may be needed. Data is collected and analyzed and utilized to improve quality of the client program in the launch to first year module. In one embodiment, Administrator Orientation (orientation education) may be a two day in-person session to provide an administrative overview and a forum for the first organization's administrators to connect. Topics include: the roles and responsibilities of the first organization's administrators, operations and sustainability, high quality program implementation, program development, policy and government affairs, strategic relations, finance, marketing, and nursing. In one embodiment, the Initial Education Unit 1 consists of approximately 30 hours of distance education covering fundamentals of nursing practice including the theoretical concept and practical application of the model, on-line modules, self-assessment tools and a self-guided study module. Unit 1 is completed and verified by the first organization prior to attendance of Units 2 or 3. Unit 2 is a 3-day, in-person session for supervisors to reinforce and deepen knowledge and skill of the topics introduced in Unit 1. Topics include motivational interviewing, six domains of the maternal role and a study of, for example, the NFP Visit-to-Visit Guidelines®, among others. Unit 3 consists of on-line individual lesson modules covering early emotional development and fidelity to the Model. Unit 4 is distance education, completed six months after core education and includes the following topics: using reports to monitor practice, building caseload and client retention.

Aspects of the launch to first year module 400 are illustrated in the flow chart of FIG. 4. As shown in FIG. 4, the launch to first year module includes development of a first year annual plan, development of a year one implementation report and administrative task completion. Data is collected and analyzed and utilized to improve quality of the client program in the year two module. Data for Program Improvement from the launch to first year module is collected, analyzed and utilized to improve quality of the agency's program implementation and/or the first organization's program quality framework. The purpose of the Annual Plan is to document the efforts that the nurse consultants and agency team are making to maintain and improve fidelity to the model. In one embodiment, the plan focuses on a minimum of three program outcomes, interventions, or implementation indicators over the specified time period. Depending on the nurse consultant's judgment and discussions with nurse supervisors, the agency team may focus on only one issue at a time. In one embodiment, the purpose of the Year One Implementation report is to provide the implementing agency and the nurse consultant with information about milestones in the implementation process. This report will enable the nurse consultant to assess the implementing agency's progress in the hiring and education of nurses, recruitment of clients and building caseloads. The nurse consultant and nurse supervisor are able to compare the agency's progress with outcomes in similar agencies In one embodiment, the Administrative Task Completion report is available as a desktop report for agency staff to insure that specific tasks needed to implement the model are completed in a timely manner. Data that will populate the report are entered by the agency staff into a web-based software system.

Aspects of the year two module 500 are illustrated in the flow chart of FIG. 5. As shown in FIG. 5, the year two module 500 includes giving surveys and/or establishing reports in the following main categories: Client Interaction, Program Implementation and Outcome Achievement. In one embodiment, for the Client Interaction sub-module, the following surveys are given: a nursing practice assessment, a client survey and a nurse supervisor assessment. The surveys that comprise this sub-module are web based and are completed by the home visitor, clients and nurse supervisor. Data from these surveys are used to measure and rate the nurse's and nurse supervisor's competency in the nursing model and the client survey measures the perception of the nurse's ability to deliver the model. The ratings are used for professional development of the nurse and nurse supervisor. Aggregate results are used by the first organization to strengthen the program in the areas of nursing education and consultation. Scoring of this sub-module may be based on the completed number of surveys. With respect to the Program Implementation sub-module, in one embodiment, the following surveys and reports are given: a critical structure elements assessment and a nurse consultant assessment. The following tasks are also completed and evaluated: implementation of an annual plan, a fidelity report and administrative task completion. The surveys, reports and assessments in this sub-module are designed as process measures. Each tool is used to measure and evaluate activities at the agency level that have been identified as strong indicators for successful implementation of the model and to report the agency's ability to meet model fidelity. Agency results will be compared with benchmarks and a score assigned. With respect to the Outcome Achievement sub-module, in one embodiment, the following outcomes and benchmarks are monitored: pregnancy outcomes, maternal outcomes, child health and development outcomes and federal home visitation initiative benchmarks. The Outcome Achievement sub-module provides evaluation of the program outcomes. Data is collected throughout the home visitation program in each of these domains. Results will be compared with benchmarks, targets and goals to assign a score in each of the domains. Data is collected and analyzed and utilized to improve quality of the client program in the year three and beyond module. Based on the scores for each tool in the quality framework, program improvement activities are conducted at the agency level in collaboration with the nurse consultant. The components of the quality improvement model include: “assess”-“analyze”-“plan”-“implement”-“measure”-“reassess”. Quality improvement techniques such as those described by Edward W. Deming (Deming, E. W. (1986), Out of Crisis, MIT Center for Engineering Study): “plan”-“do”-“study”-“act” are included in nursing education and ongoing consultation. Reports for the quality framework are analyzed and utilized to improve quality of the agency's program implementation and/or the first organization's overall program quality (i.e. data for Program Improvement from the year two module is input back into the model).

Aspects of the year three and beyond module 600 are illustrated in the flow chart of FIG. 6. As shown in FIG. 6, in one embodiment, the year three and beyond module 600 includes giving surveys and/or establishing reports in the following main categories: Client Interaction, Program Implementation and Outcome Achievement. For the Client Interaction sub-module, the following surveys are given: a nursing practice assessment, a client survey, a nurse supervisor assessment and a nurse consultant assessment. The surveys that comprise this sub-module are web based and are completed by the home visitor, clients and nurse supervisor. Data from these surveys are used to measure and rate the nurse's and nurse supervisor's competency in the nursing model and the client survey measures the perception of the nurse's ability to deliver the model. The ratings are used for professional development of the nurse and nurse supervisor. Aggregate results are used by the first organization to strengthen the program in the areas of nursing education and consultation. Scoring of this sub-module may be based on completed number of surveys. With respect to the Program Implementation sub-module, the following survey and reports are given: a critical structure elements assessment and the Nurse Consultant Assessment. The following tasks are also completed and evaluated: implementation of an annual plan, a fidelity report and administrative task completion and a periodic implementation review. The surveys, reports and assessments in this sub-module are designed as process measures. Each tool is used to measure and evaluate activities that have been identified as factors for successful implementation and to report the agency's ability to meet model fidelity. Agency results will be compared with benchmarks and a score assigned. With respect to the Outcome Achievement sub-module, the following outcomes and benchmarks are monitored: pregnancy outcomes, maternal outcomes, child health and development outcomes and federal home visitation initiative benchmarks. The Outcome Achievement sub-module provides evaluation of the program outcomes. Data is collected throughout the home visitation program in each of these domains. Results will be compared with benchmarks, targets, and goals to assign a score in each of the domains. Data is collected and analyzed and utilized to improve quality of the client program in the year three and beyond module (e.g. data from year three is used for at least year four, data from year four is used for at least year five and so on). Based on the scores for each tool in the quality framework, program improvement activities are conducted at the agency level in collaboration with the nurse consultant. The components of the quality improvement model include: “assess”-“analyze”-“plan”-“implement”-“measure”-“reassess”. Quality improvement techniques such as the Deming model of “plan”-“do”-“study”-“act” are included in nursing education and ongoing consultation. Data for Program Improvement from the year three and beyond module is collected, analyzed and utilized to improve quality of the agency's program implementation and/or the first organization's program quality framework. More specifically, reports for the quality framework are analyzed and utilized to improve quality of the agency's program implementation and/or the first organization's overall program quality.

FIG. 7 is a flow chart illustrating aspects of the data analysis and adjustment module 700 utilized by the method disclosed herein and aspects of which may be performed by a computer system 800 as disclosed herein. As indicated in FIG. 7, raw data from the previous modules is input into the system and analyzed. Each of the sub-modules (Client Interaction, Program Implementation and Outcome Achievement) has a certain total point value that may be awarded. The total points awarded are sorted and weighted and reported as a final agency score. A total score between 270 and 300 indicates a high performing agency, no improvement is indicated and the agency proceeds to the next year on track. A total score between 151 and 269 indicates a performing agency, improvement is indicated in order to become a high performing agency, but the agency may continue to the next year on track. A total score between 31 and 150 indicates a low performing agency, improvement is required and development and implementation of an improvement plan is required to continue into the next year. A total score between 0 and 30 indicates a critically low performing agency.

The first organization may review closure criteria for the agency and if closure is not recommended, improvement is required and development and implementation of an improvement plan is required to continue into the next year. If closure is recommended, in one embodiment, the first organization may terminate the implementation contract and the agency's license to implement the quality framework program detailed herein.

FIG. 8 is a flow chart illustrating one embodiment of a system 800 for data analysis which may be used in accordance with the method of the present disclosure. As shown in FIG. 8, embodiments within the scope of the invention include computer systems 800 configured to perform data analysis in accordance with the methods disclosed herein and, in some embodiments, produce a graphical representation thereof. In one embodiment, a computer system for analyzing survey results comprises inputting raw data from survey results (e.g. from assessments made/given in conjunction with modules: “launch to first year”, “Year Two” and “Years Three and Beyond” (see e.g. FIGS. 1, 4, 5 and 6) according to the method disclosed herein.

Computer systems are generally well-known in the art. Those skilled in the art will appreciate that aspects of the invention may be practiced in computing environments or network computing environments with many types of computer system configurations, including personal computers, hand-held devices, multi-processor systems, microprocessor based or programmable consumer electronics, network PCs, minicomputers, mainframe computers, and the like. Further, wirelessly connected cell phones, a type of hand-held device, are considered as within a network computing environment. For example, cell phones include a processor, memory, display, and some form of wireless connection, whether digital or analog, and some form of input medium, such as a keyboards, touch screens, etc.

Examples of wireless connection technologies applicable in various mobile embodiments include, but are not limited to, radio frequency, AM, FM, cellular, television, satellite, microwave, WiFi, blue-tooth, infrared, and the like. Hand-held computing platforms do not necessarily require a wireless connection. For example, a hand-held device may access multimedia from some form of memory, which may include both integrated memory (e.g., RAM, Flash, etc) as well as removable memory (e.g., optical storage media, memory sticks, flash memory cards, etc.) for playback on the device. Aspects of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

FIG. 8 illustrates the components of a computer system 800 that may be configured to perform data analysis which may be used in accordance with the method of the present disclosure. The computer system 800 may include a user interface 802, memory 804, a processor 806, raw data 808, such as data from the surveys indicated in FIGS. 1 and 4-6, a client interaction process 810, a program implementation process 812 and a outcome achievement process 814. Outputs may include a numerical output 816 identifying results/total scores from the sorting and weighting operations which occur within each process 810, 812, 814. While each process 810, 812, 814 identifies specific reports, surveys and assessments for analysis, it can be appreciated that other or additional reports, surveys and assessments as described elsewhere herein may be analyzed in a given process and/or fewer reports, surveys and assessments may be analyzed in a given process.

In certain embodiments, and as can be understood from FIG. 8, computer systems 800 include a processor 806 configured to perform the methods disclosed herein and capable of executing program instructions. Accordingly, the processor 806 may include any general purpose programmable processor or controller for executing application programming. Alternatively, the processor 806 may comprise a specially configured application specific integrated circuit (ASIC). The processor 806 generally functions to run a programming code implementing various functions performed by the processes 810, 812, 814 or other system component being implemented. For example, such functions may include functions enabled through the execution of programming code or other application instructions.

The computer system 800 may additionally include memory 804 for use in connection with the execution of programming by the processor 806, and for the temporary or long term storage of data or program instructions. For example, the memory may be used in connection with the operation of applications. The memory 804 may comprise solid-state memory resident, removable or remote in nature, such as DRAM and SDRAM and as described previously. Examples of particular applications that may be stored in the memory 804 include: a client interaction process 810, a program implementation process 812 and a outcome achievement process 814. The raw data 808 that may be input into the system includes the survey data from FIGS. 1 and 4-6. Such raw data may include a data set of raw data. The data may be input manually or stored in the memory of the computer system.

Embodiments within the scope of the present invention also include computer readable media for carrying or having computer-executable instructions or data structures stored thereon. Such computer-readable media may be any available media that can be accessed by a general purpose or special purpose computer. By way of example, and not limitation, such computer-readable media can comprise RAM, ROM, EEPROM, DVD, CD ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications link or connection (either hardwired, wireless, or a combination of hardwired or wireless) to a computer, the computer properly views the connection as a computer-readable medium. Thus, any such connection is properly termed a computer-readable medium.

Combinations of the above should also be included within the scope of computer-readable media. Computer-executable instructions comprise, for example, instructions and data which cause a general purpose computer, special purpose computer, or special purpose processing device to perform a certain function or group of functions. In one embodiment, a computer readable medium including computer executable instructions to, when implemented, perform the methods described herein, such as the method for weighting and sorting data from program surveys and assessments.

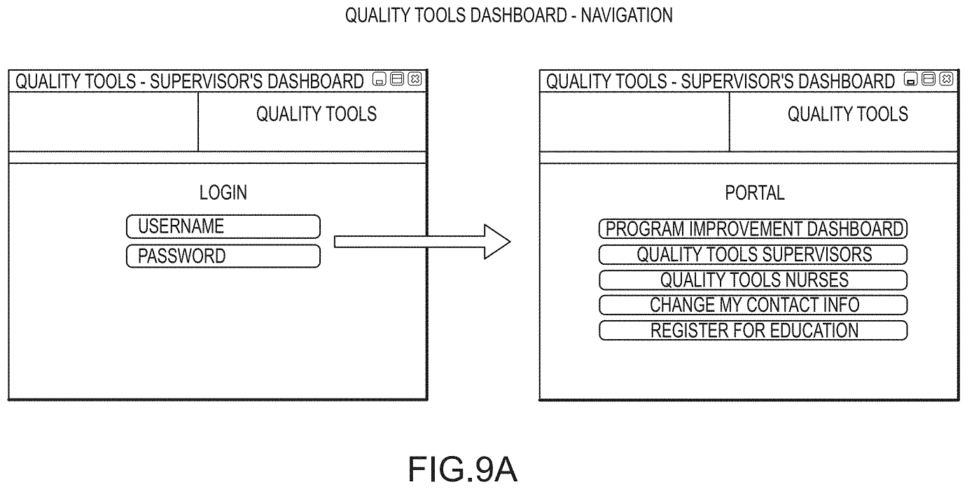

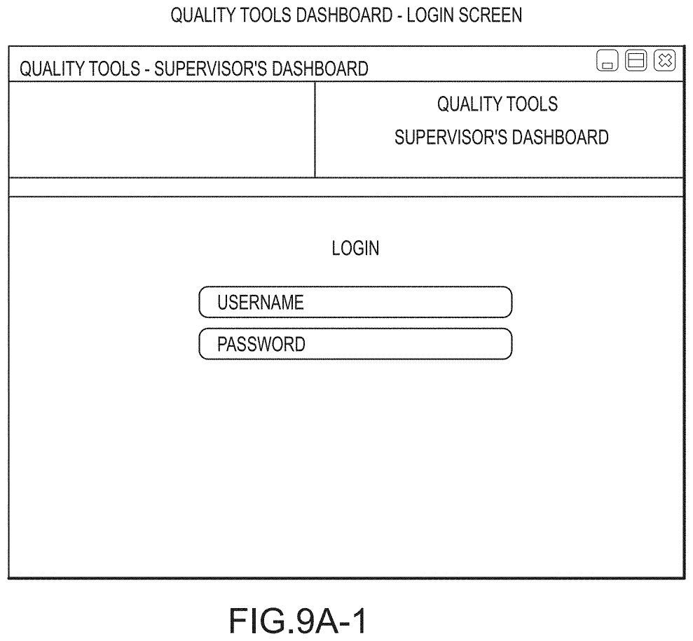

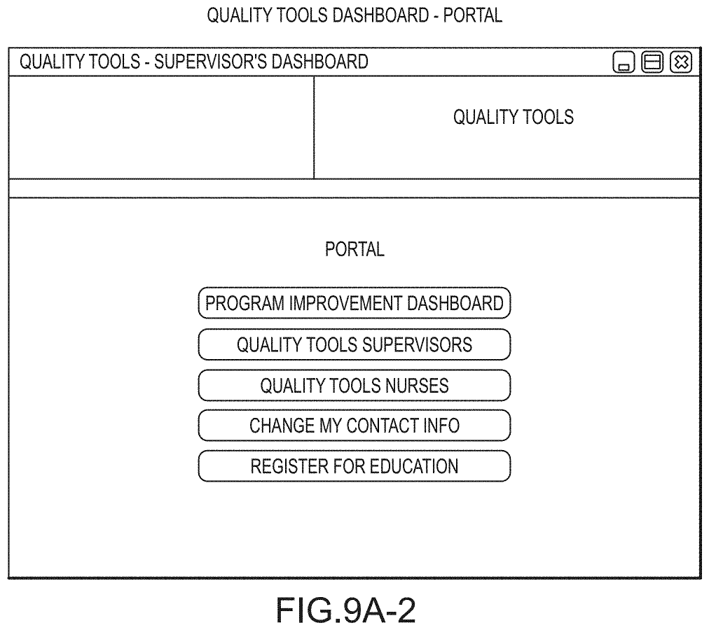

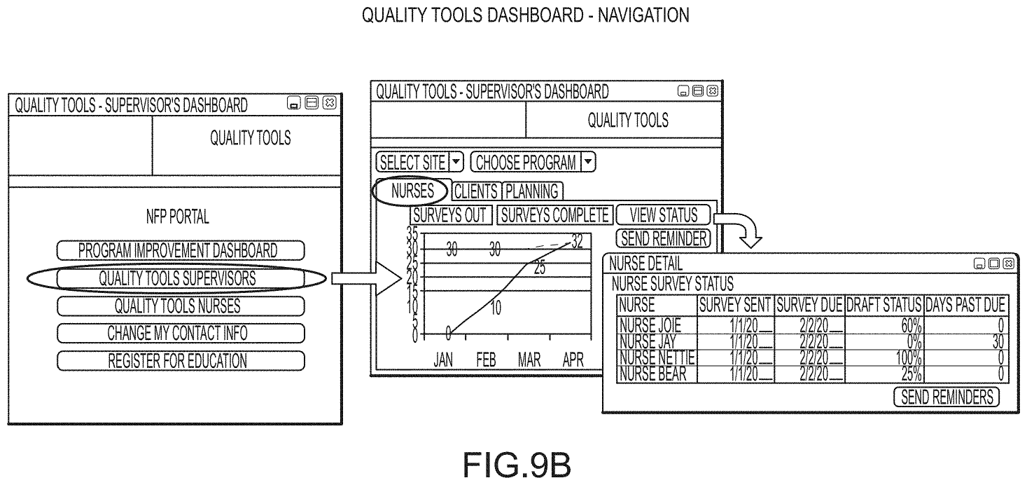

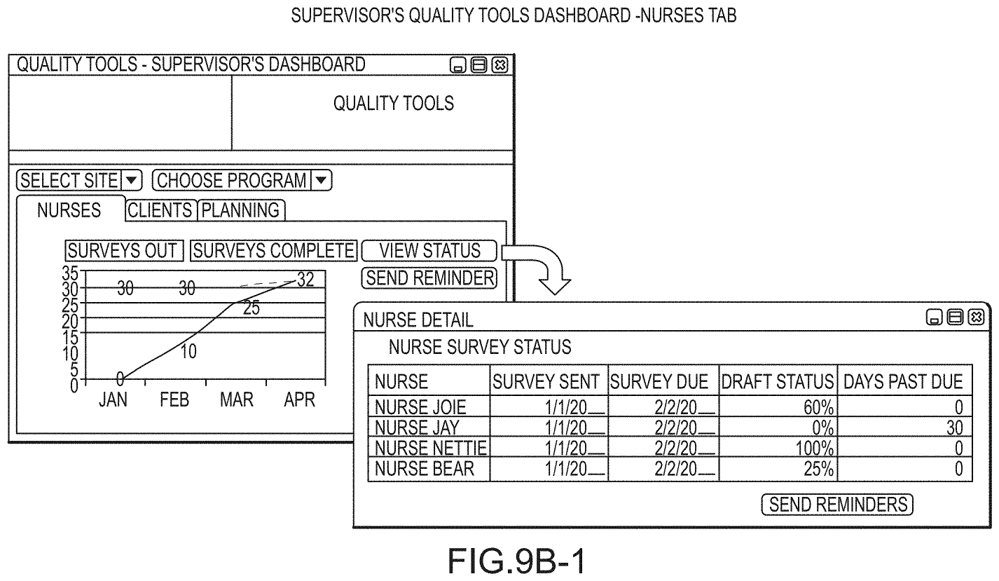

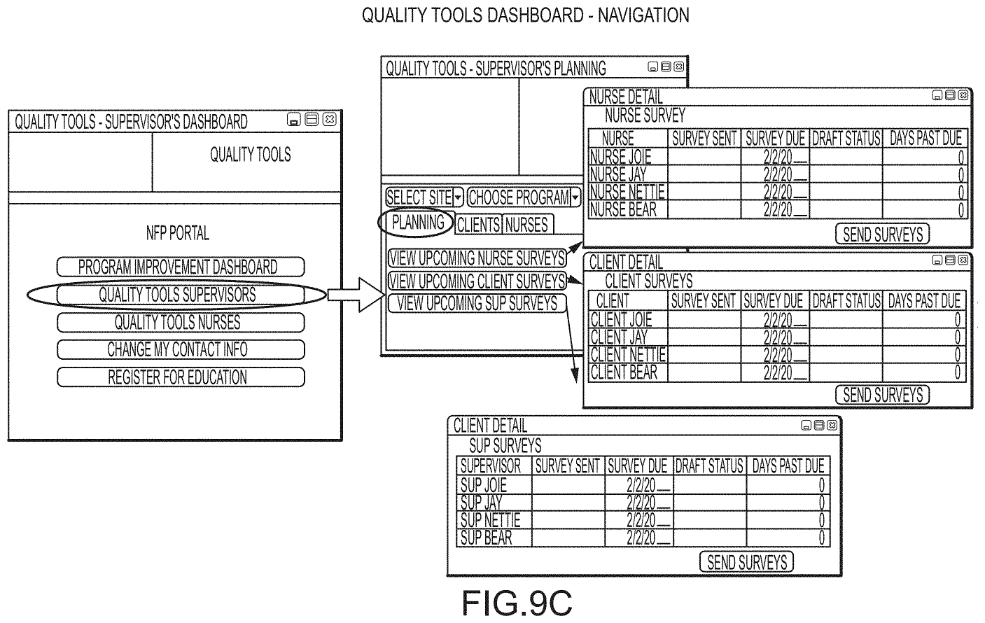

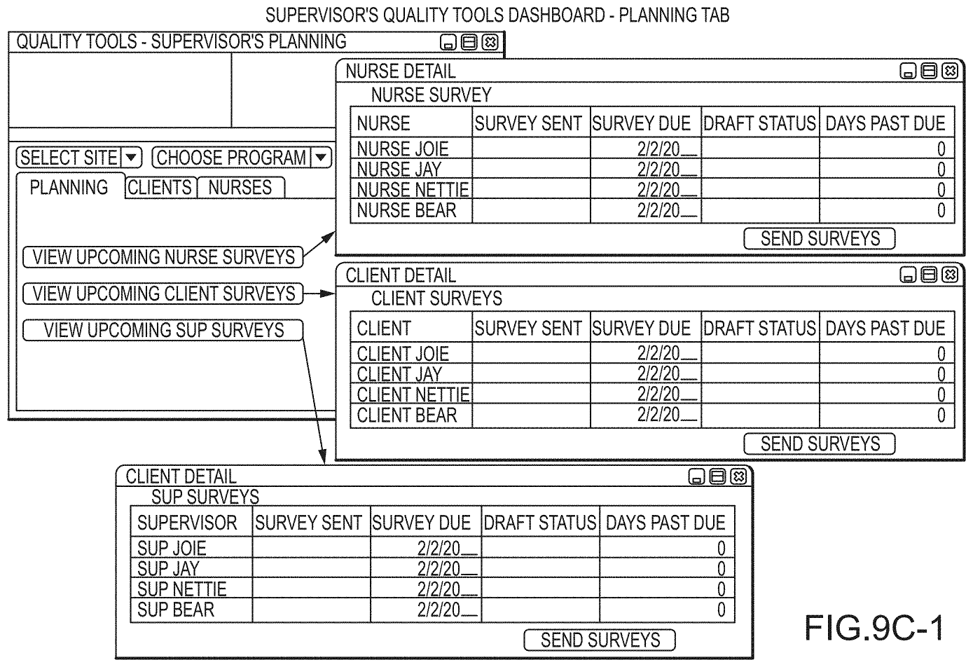

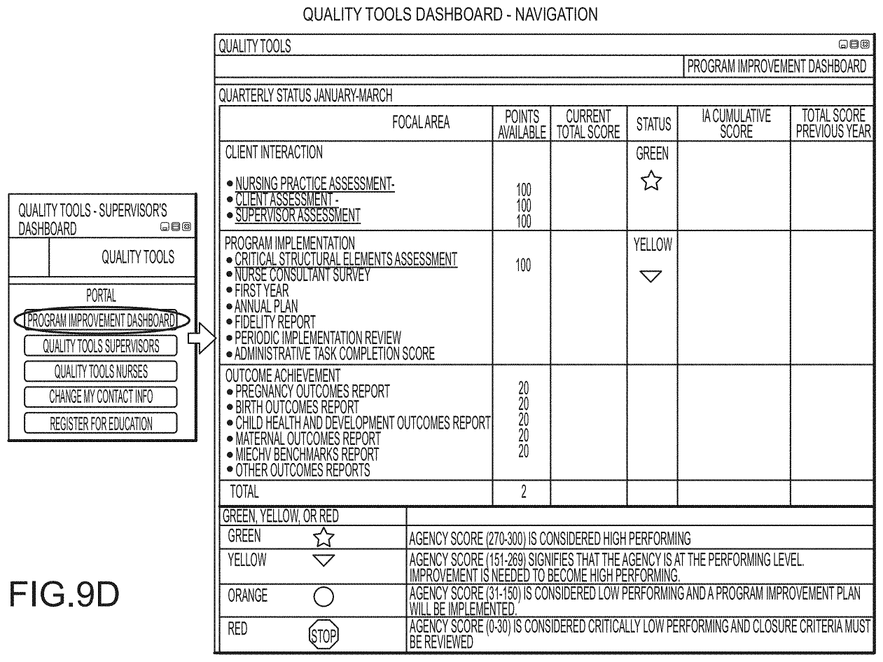

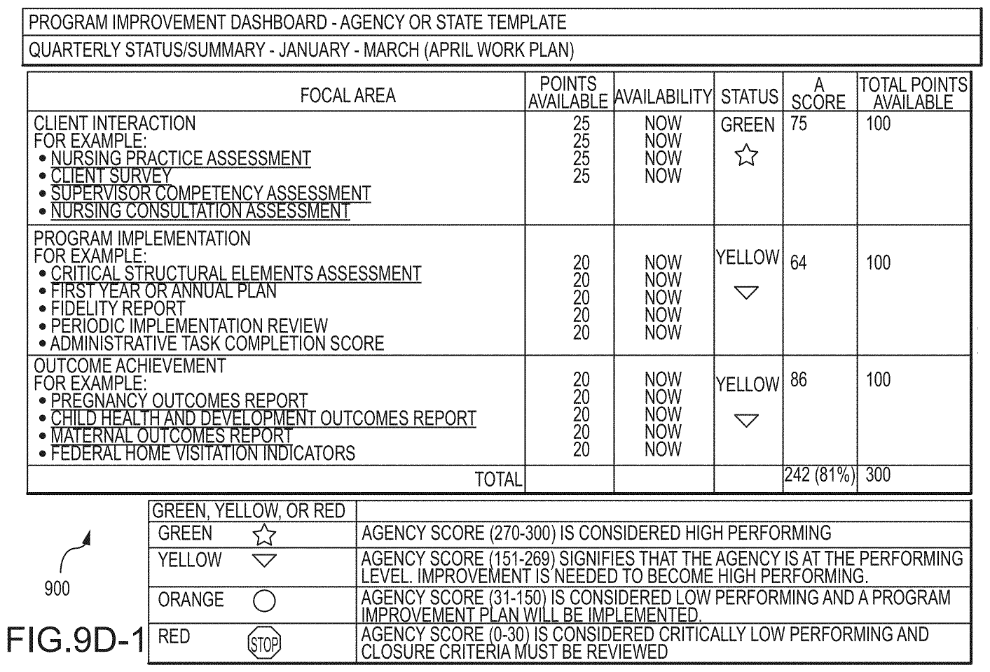

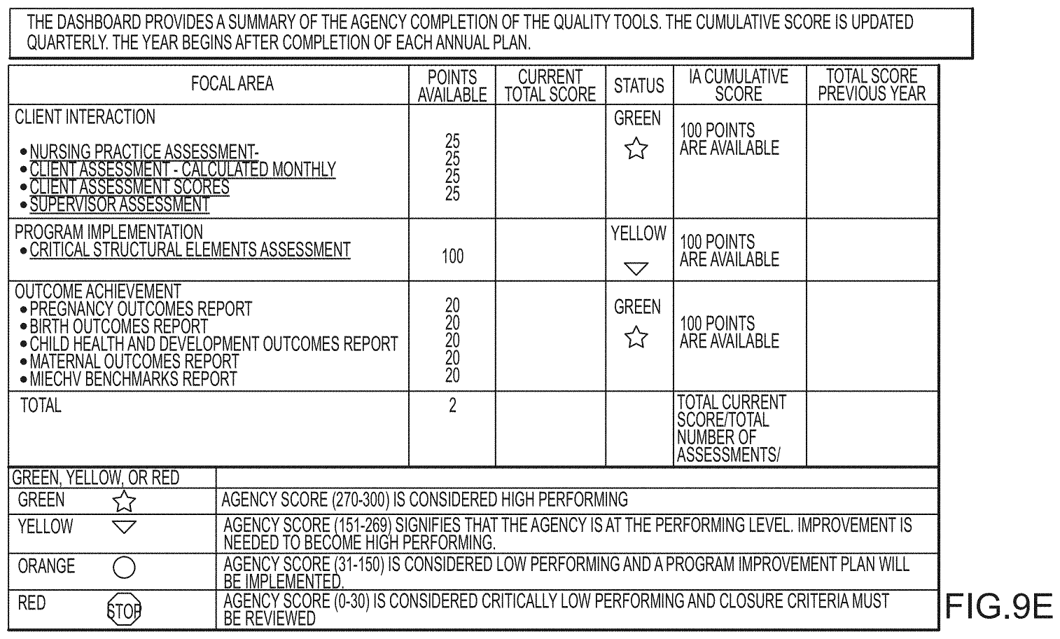

In some embodiments, the computer implemented method may further comprise producing a numerical or graphical representation or output thereof or a user interface, such as the user interfaces/outputs/report dashboards illustrated in FIGS. 9A-9E. FIGS. 9A-9E depict exemplary dashboards that may be used with the system 800. The dashboards can be accessed by an agency, its employees or the first organization or its employees to quickly review status and progress of an individual agency. (See e.g., the initial login and portal screens of FIGS. 9A and 9A-1 and 9A-2 (an expanded views of 9A). The dashboards are populated from the enterprise data warehouse based on the assessments and surveys listed earlier. The dashboards as a performance strategy translates the goals of the quality framework into a format that will enable the users to monitor and measure their outcomes and will support continuous quality improvement. The dashboards communicate the goals, supports coordination between the implementing agency and the first organization, and provides consistent information across the organization. As depicted in FIGS. 9A-9E, a series of dashboards are available to the end user for the purpose of monitoring each assessment or report in real-time. In addition to the dashboards, the system will generate quarterly reports, such as the Quality Summary Dashboard (see FIG. 9E). This report will summarize each of the products of the quality framework and the scoring.

As indicated in the previous figures, scoring of the tools and assessments includes measuring the agency's ability to complete the assessments in a timely manner and/or ranking the agency's performance in meeting the expectations of the assessments.

As discussed in more detail below with respect to FIGS. 9A-9E, in one embodiment, a series of dashboards are used by the agency to monitor their performance in completing the client survey, the nurse practice assessment and the nurse supervisor assessment. In some embodiments, a quarterly report using the Quality Summary Dashboard (see, e.g. FIG. 9E) provides a snapshot of success in each area.

As indicated above, the surveys, assessments and other tools are completed and the responses weighted and scored. In various embodiments, scoring or weighting may be different based on whether the agency is located in a rural or urban site or whether the agency is a new or established organization. In one embodiment, scoring and weighting of each process includes the following analyses (see e.g. FIGS. 9D, 9D-1 (an expanded view of 9D) and 9E):

1) The Client Interaction Process includes (a) Client Survey. Monthly, the agency is notified of the clients to receive a client survey. The dashboard monitors the completion of the assessment and quarterly a percent completed is listed in the Quality Summary Dashboard. Agencies are expected to attain a 50% return rate to be considered functioning at the performing level. A score less than 25% will indicate poor performing. The final score is calculated annually. Agencies are also expected to attain a scoring of 2 or greater on each client survey. This scoring is averaged quarterly and the final score is determined annually. (b) Nursing Practice Assessment and the Nurse Supervisor Assessment. Annually, in collaboration with the Nurse Consultant, an agency agrees to complete the NPA and NS for each current staff member. The dashboard monitors compliance with the individual team members. (See e.g., FIGS. 9B, 9B-1 (an expanded view of 9B), 9C, 9C-1 (an expanded view of 9C). The Quality Summary Dashboard provides a quarterly summary of the percent completed. The scores for each tool in the client survey process are equally ranked.

2) The Program Implementation Process includes the Critical Structural Elements, the Fidelity Report, the Periodic Implementation Review and Administrative Task Completion. The Critical Structural Elements (CSE) is evaluated annually for completion. The CSE is a series of reports that identify if the agency is performing as designed. There are 5 reports, each of which will need to be completed at 100%. The scoring will be annually, the Quarterly Summary Dashboard will provide a percent quarterly. In one embodiment, only the CSE is scored and the points available is 100. Agencies that rank 90-100 will be considered high performing; fewer than 70% will be considered low performing and below 50% will be considered critically low performing. In other embodiments, additional tools are scored and weighted such that the total number of points available (100) is divided evenly between each tool. For example, in an embodiment where both the CSE and the Fidelity Report are analyzed, the total number of points (100) is divided by 2, such that each tool has a total of 50 points available. Each tool is subject to benchmarking and scoring. The scores for each tool in the program implementation process are equally ranked.

3) The Outcome Achievement Process includes pregnancy outcomes report, maternal outcomes report, child and health development outcomes report and federal home visitation indicators report. Analysis of the outcome reports has determined which influencing factors impact outcomes. Based on this comparison, each tool is benchmarked. Scoring is determined based on benchmark scores. For instance, an agency may score between 85-100 in the various reports; this may reflect a high performing agency. There are several aspects of each report, each will be analyzed and benchmarked. The scores for each tool in the outcome achievement process are comparably weighted.

All directional references (e.g., proximal, distal, upper, lower, upward, downward, left, right, lateral, front, back, top, bottom, above, below, vertical, horizontal, clockwise, and counterclockwise) are only used for identification purposes to aid the reader's understanding of the present invention, and do not create limitations, particularly as to the position, orientation, or use of the invention. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. The exemplary drawings are for purposes of illustration only and the dimensions, positions, order and relative sizes reflected in the drawings attached hereto may vary.

The above specification, examples and data provide a complete description of the structure and use of exemplary embodiments of the invention as claimed below. Although various embodiments of the invention as claimed have been described above with a certain degree of particularity, or with reference to one or more individual embodiments, those skilled in the art could make numerous alterations to the disclosed embodiments without departing from the spirit or scope of this invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A method for implementing a quality control and improvement program in an evidence-based organization, the method comprising
evaluating an implementation plan and/or feasibility assessment;
developing an annual plan by incorporating findings from the evaluating operation into the plan;
applying the annual plan within the evidence-based organization;
providing assessment surveys and/or reports to stakeholders of the evidence-based organization, wherein the stakeholders comprise at least one of a first-time mother and a nurse;
collecting raw data from the assessment surveys; and
analyzing the raw data from the assessment surveys to identify action items for improvement for the stakeholders and further to identify action items for improvement for the evidence-based organization as a quality control program.

2. The method of claim 1, further comprising implementing the action items for the stakeholders.

3. The method of claim 1, further comprising implementing the action items for the evidence-based organization as a quality control program.

4. The method of claim 1, further comprising providing training after the evaluating operation.

5. The method of claim 1, wherein said analyzing operation comprises
analyzing raw data from the assessment surveys by determining a score for each response in the survey; and
sorting and weighting each response score to determine an overall score.

6. The method of claim 5, wherein the overall score is evaluated and used to develop and implement the quality control program for the evidence-based organization.

7. The method of claim 1, wherein the evidence-based organization is a home visitation organization established to help low-income, first time mothers, pregnant mothers, infants, young children and home visitors.

8. The method of claim 1, wherein the assessment surveys include at least one of client interaction surveys, program implementation surveys and outcome achievement surveys.

9. The method of claim 1, wherein the implementation plan includes data related to at least one of: (i) characteristics of a community to be served by the evidence-based organization; (ii) budget and funding mechanisms; (iii) a timeline for implementation; and (iv) identification of challenges expected by the evidence-based organization in its implementation.

10. The method of claim 1, wherein the feasibility assessment includes data related to at least one of: (i) identification of one or more needs of a community to be served by the evidence-based organization; (ii) evidence of a viable client referral network; and (iii) presence of community resources to support referrals made by the evidence-based organization.

11. The method of claim 5, wherein the analyzing operation further comprises comparing the response score or the overall score to a benchmark or a target score.

12. The method of claim 5, further comprising evaluating the overall score by determining where the score falls in a range of scores, wherein the range provides an indication of a performance level of the evidence-based organization or the stakeholders.

13. The method of claim 12, wherein the performance level is high performing, performing, low performing or critically low performing.

14. The method of 13, further comprising developing and implementing an improvement plan for an evidence-based organization or stakeholders having a performance level of low performing or critically low performing.

15. A system for implementing a quality control and improvement program in an evidence-based organization, the system comprising
a computer, the computer comprising
a memory for storing computer readable code; and
a processor operatively coupled to the memory, the processor configured to:
receive raw data from assessment surveys given to stakeholders of the evidence-based organization, wherein the stakeholders comprise at least one of a first-time mother and a nurse;
analyzing the raw data from the assessment surveys to determine a score for each response in the survey; and
sorting and weighting each response score to determine an overall score,
wherein: the overall score is utilized to identify action items for improvement for the stakeholders and further to identify action items for improvement for the evidence-based organization as a quality control program.

16. The system of claim 15, wherein the computer is a special purpose computer.

17. The system of claim 15, wherein the evidence-based organization is a home visitation organization established to help low-income, first time mothers, pregnant mothers, infants, young children and home visitors.

18. The system of claim 15, wherein the assessment surveys include at least one of client interaction surveys, program implementation surveys and outcome achievement surveys.

19. The system of claim 15, wherein the analyzing operation further comprises comparing the response score or the overall score to a benchmark or a target score.

20. The system of claim 15, further comprising evaluating the overall score by determining where the score falls in a predetermined range, wherein the predetermined range provides an indication of a performance level of the evidence-based organization.
